(19) United States
(12) Patent Application Publication
(10) Pub. No.: US 2010/0016679 A1
(43) Pub. Date: Jan. 21, 2010

(54) Apparatus and Method for the Identification of a Subject Memory Trace and for the Intention Prevision

(75) Inventors: Giuseppe Sartori, Mestre (IT); Sara Agosta, Gazzuolo (IT)

Correspondence Address: SHOEMAKER AND MATTARE, LTD, 10 POST OFFICE ROAD - SUITE 100, SILVER SPRING, MD, 20910, US

(73) Assignee: Universita' Degli Studi di Padova, Padova (IT)

(21) Appl. No.: 12/504,027

(22) Filed: Jul. 16, 2009

(30) Foreign Application Priority Data
Jul. 17, 2008 (IT) PD2008A000211

(51) Int. Cl. A61B 5/16

(52) U.S. Cl. 600/300

(57) ABSTRACT

An apparatus and method for the identification of a subject's memory trace and for intention prediction use a procedure for acquiring information by means of administering to the subject a series of tests; the apparatus records the answers and the reaction times of the subject and it elaborates the results in order to give, with an accuracy of 92%, the memory trace or the intention of the subject. The apparatus and method give the possibility to avoid or identify maligners and fakers.

### FIELD OF THE INVENTION

This invention regards an apparatus and a method for the identification of a memory trace and the prediction of a subject's intention. It is also suitable for “lie-detection” or “intention-detection” applications in fields such as: security, intelligence, investigation, marketing and work selection.

### BACKGROUND OF THE INVENTION

Numerous different methods for detecting lies are known, but the most know lie-detector usually refers to the “polygraph”.

The “lie detector” or polygraph is an apparatus that continuously measures different physiological responses (changes in respiration, electrodermal activity, blood pressure, perspiration, brain potentials) while the examinee is exposed to a series of questions. Physiological responses of the subjects are measured in two conditions: in respect to “critical” questions or items, strictly associated to the object of the investigation (e.g. crime that is object of inquiry: “Have you killed your wife?”) and control questions for which the answer is known (e.g. “Have you been condemned for a theft?”). If reaction to critical and control questions are similar the subject is honest, while if the if they are different, the subject is lying. There are two main types of test that are used while measuring psychophysiological responses: the Control Question Test (CQT; Ben-Shakar, 2002) and the Guilty Knowledge Test (Lykken, 1974).

The most widely used method of psychophysiological detection of deception is the Control Question Test (CQT) where physiological responses to critical questions are compared to physiological responses control questions. The CQT typically consists of about 10 questions. As already mentioned, there are two types of questions that are important in the determination of guilt or innocence: relevant and control questions. The relevant questions refer directly of the facts under investigation (e.g., Did you shoot Mr. Bianchi on the night of 24 May?), while control questions cover past behavior that might be associated with personality traits that are related to the potential readiness of committing a crime (e.g., Before the age of 30, did you deliberately hurt someone you were close to?). It is assumed that guilty subjects will be physiologically more reactive to the relevant than to the control questions, while the reverse pattern is expected with innocent people.

The second procedure for lie detection is known as Guilty Knowledge Test (Likken, 1974), or Concealed Information Test (Lykken, 1959, 1960). It has drawn considerable attention among researchers, in fact there is an ever-growing body of literature that treat of this argument. In contrast with the CQT there is a general consensus about the scientific principles at the basis of the GKT. The GKT finalized by Lykken (Lykken, 1959, 1960) consists of a series of multiple-choice questions. Each one comprehends one relevant answer (feature of the crime under investigation) and several neutral (control) answers, chosen so that the innocent suspect would not be able to discriminate them from the relevant item (Lykken, 1998). The subject is exposed to a series of scenes or objects while emotive reactions are measured. Only the person that recognize some of these stimuli as strictly related to the crime (the crime that he/she perpetrated) show emotive reactions. The GKT uses a series of multiple-choice questions. Each one comprehend a relevant option (related to the crime) and a series of distracting stimuli, chosen so that the innocent subject cannot distinguish among them (Lykken, 1998) and only the guilty subject can recognize that relevant option. An example of a GKT question is the following: “What kinds of gun was used to shoot Mr. McCain?: a) 22-caliber rifle? b) 12-gauge shotgun? c) a 38-caliber revolver? d) an M-16 rifle? e) a 9-mm handgun?” (example mentioned by MacLaren, 2001). Knowledge about the crime-event is inferred if a suspect's physiological responses to relevant questions are consistently higher in respect to the neutral answer physiological responses.

The GKT is also used with Event Related Potentials. The P300 is a brain wave recorded trough electrodes placed on the scalp, while the suspect is presented a series of stimuli. The P300 is directly related to cognitive and memory processes involved in recognition of previously learned material, therefore it offers a sensitive and conceptually informative approach to the detection of guilty knowledge. Its amplitude increases in inverse relationship to the probability of occurrence of a stimulus, this means that the P300 is larger for stimuli that rarely occur. In a P300 GKT procedure, the crime relevant items are included among the rare stimuli. When these items are mixed together with the crime-irrelevant alternatives, the person without guilty knowledge will not recognize any of the alternatives, so none elicits the P300, but the guilty respondent will recognize the relevant items as “special” elicit a P300. We can imagine that a person has been killed using an hammer and the guilty suspect is undergoing this method. The P300 response to the picture of an hammer is different than the response related to control stimuli.

Given the low reliability of the polygraph, researchers are trying to validate new techniques that permits to identify “liars”. For this reason a number of alternatives have been proposed, under the growing necessity of a reliable lie-detector following the September 11 episode. But no one clearly outperforms the others as regards to accuracy.

A new technique for detecting lies is based on the functional magnetic resonance imaging (fMRI). The subject is scanned while he/she is asked questions regarding the crime. fMRI measures the BOLD (Blood-Oxygen-Level-Dependent) signal while the examinee in engaged in truthful and deceptive responses. Studies using fMRI started after 2000 (e.g., Langleben, Loughead, Bilker, Ruparel, Childress, Busch, & Gur, 2005). Several fMRI studies, using the GKT, reported increased prefrontal and parietal activity during lie, with some studies reporting Anterior Cingulated Cortex (ACC) and Dorsolateral prefrontal cortex (DLPFC) activation (e.g., Ganis, Kosslyn, Stose, Thompson, & Yurgelun-Todd, 2003). Based on these findings, deception seems to involve the inhibition truthful responses as well as the production of a lie and the frontal network is involved with truthful response being a “default” response. In particular, the DLPFC is related to the maintenance of information in working memory while credibility of the lie. By contrast, the ACC is responsible of the inhibition of the truthful response fMRI studies use, for the most part. The GKT technique has been used in order to identify the neural correlates of the inhibition process and the suppression of the automatic answer (Langlebeen et al., 2002). Participants, for example, are typically asked to select one of two cards and then instructed to lie about his/her selection. Despite the use of different paradigms the activation of two areas (DLPFC and ACC) is typically reported. These results highlight the fact that “lying” is a complex cognitive process and involves, depending on the lie cognitive complexity, many high-level functions, such as: decision-making, response monitoring, and attribution of mental states. At first sight, it is possible to think that there is a specific activation for “lying”, but the problem arises from the fact that the same areas are activated during different tasks that involve the same cognitive processes, without lying.

Another new technique is based on the use of the Infrared. This technique is used to measure body heat emissions (Pollina, Dollins, Senter, Brown, Pavlidis, Levine & Ryan, 2006). Infrared Facial Image Analysis (IFIA) spots liars on the basis of thermal modifications in the periorbital regions. Studies from Pavlidis and colleagues (2006) showed that the periorbital area is the “area mostly affected by the blood flow distribution during anxious states”. This technique may be used with both CQT or GKT paradigms.

All these techniques present several disadvantages. These techniques have been used since their development more than 80 years ago but the Committee to Review the Scientific Evidence on the polygraph (Washington, 2003), concluded that it lack in reliability and validity. First of all, these methods are sensitive to effective countermeasures consisting in the use of physical (biting the tongue) and mental (count backward by 7) techniques (Honts, Raskin and Kirker, 1994). Second, the dependent measure under consideration is the emotional reaction connected to the lie, the “anxiety” is not necessarily caused by the lie (Wolpe, Forster, Langlebeen, 2005). The physiological reaction is not uniquely related to the lie and can also be caused by “fear” as well as “stress” or “embarrassment”. In other terms, in some occasions, the innocent suspect could show the same physiological reactions of a guilty suspect (Canli, Brandon, Crowley, DuRousseau, Greely, Pascual-Leone, 2007).

fMRI studies show that lies may be distinguished from truthful responses at group level. However, generalization of these results to single subjects may not be straightforward and, at present, has not been shown to be valid. Diagnostic at individual level may be difficult to to individual differences such as brain morphology, motivation, training, mood, personality, and unknown variables. More studies are necessary in order to disentangle critical questions as the individual activation (langlebeen ate al., 2005) and the ecological validity or the administration to subjects with mental problems or that committed criminal activities, in fact their deceptive abilities can be different (Wolpe et al., 2005). Other limits are directly related to the paradigm used with polygraph and fMRI based lie detection techniques (CQT and GKT), first of all the ecological validity. In lab experiments, usually participants are instructed to lie. Given that, by definition deception is an interactive process that request a victim (Vrji, 2004), the paradigms used in the laboratories cannot have easily generalize to the real settings. The internal validity is related to the capacity of the method to control confounding variables, it comprehend the design, data collection and data analysis. The design of an experiment is related to: paradigms (GKT or CQT), risks for the participants, scenarios. In conclusion it is possible to highlight the fact that the new technologies have not been able to solve problems related to the use of CQT and GKT. Numerous problems have to be solved and it is necessary to stress the fact that lying involve a social dimension and has to be studied as a social dimension and that the action of lying can take place without an explicit lie.

### SUMMARY OF THE INVENTION

The objective of the invention is to solve the problems above highlighted. All these problems are solved by the apparatus according to claim 1″ and by a method according to claim 4. Other forms of use of the same invention are described in the other claims.

### BRIEF DESCRIPTION OF THE DRAWINGS

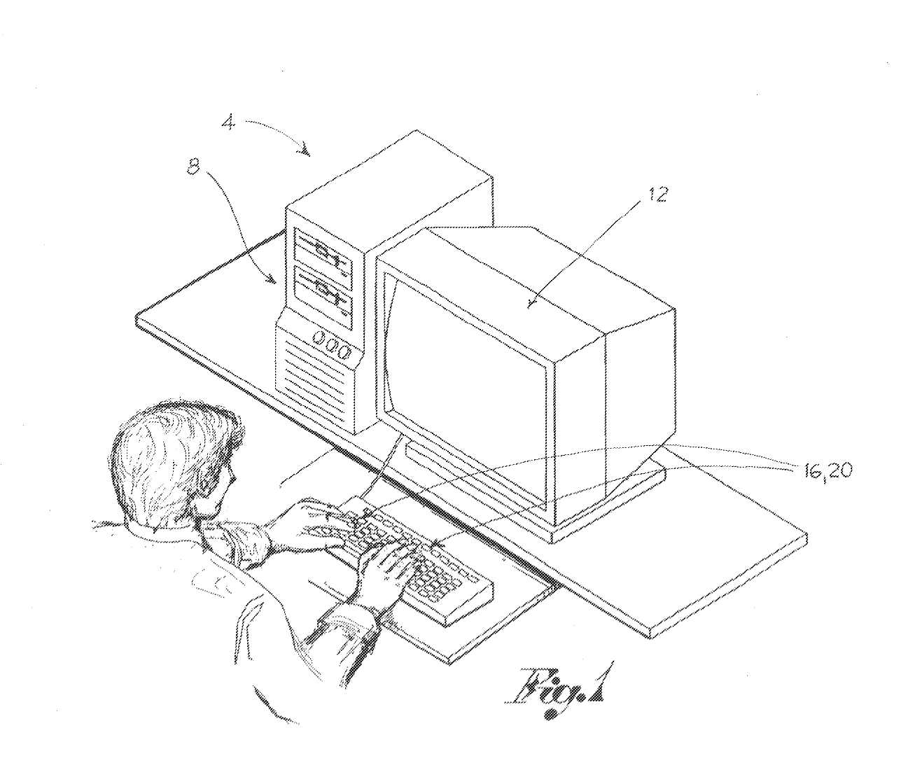

Other characteristics and advantages of the present invention will be highlighted in the description in which some examples are also reported, these have to be considered as examples and non-exclusive applications, wherein FIG. 1 shows the apparatus according to an embodiment of the present invention.

### DETAILED DESCRIPTION OF THE INVENTION

The method used here is an innovative technique based on the Implicit Association Test (IAT). The standard Implicit Association Test (Greenwald, Mc Ghee, & Schwartz, 1998) offers a method for indirectly measuring strengths of associations between concepts on the basis of the reaction times. In the IAT, items related to four concepts are presented in a random order and participants have to classify them in the respective category but, instead of requiring four kind of responses, they require only two reactions by the subject, so two categories require a response and other two categories another response. The underlying assumption is that if two strictly associated concepts require the same response and other two concepts another response, individuals are able to react faster than if the two closely associated concepts require different reactions. In fact, in this case responses should be relatively slow. The IAT effect is the difference in reaction times in these two blocks, the block that requires two different responses for the two associated concepts (termed as Incongruent Block) and the block where the associated concepts require the same response (termed as Congruent Block). The IAT evaluates the association between a target-concept discrimination and an attribute dimension. The IAT is composed of five tasks. An example of the IAT from Greenwald and colleagues (Greenwald et al., 1998) is described in the following paragraph.

In this example the target-concept discrimination refers to “White-Black”, and the attribute is “pleasant-unpleasant”. The procedure starts with introduction of the target-concept discrimination, in which participant have to distinguish first name that are recognizable as Black or Afro-American from ones recognizable as White or European-American. This and the subsequent tasks are performed assigning one response to the left hand and the other to the right hand. For example, in the first block the subject is requested to press the “A” key if a typical Black (Afro-American) picture appears, while they have to press the “L” key if a typical White (European-American) picture appears. The second task requires the classification of the attribute, categorizing words as pleasant and unpleasant in meaning. In this case subject have to press the key “A” for “pleasant” words, while they have to press the key “L” for “unpleasant” words. The two tasks are superimposed in the third step in which stimuli for target and attribute discrimination are presented on alternate trials. In this case participants have to press the key “A” for Black pictures and “pleasant” words, and the key “L” for White pictures and “unpleasant” words. In the fourth step the participant learns a reversal of response assignment for the target discrimination, so he/she is requested to press “A” for White pictures, and “L” for Black pictures. In the fifth task there is the combination of the attribute discrimination with the reversed target discrimination, subjects are requested to press the key “A” if White pictures or pleasant words appear, and to press “L” if Black pictures or unpleasant words appear. If the target categories are differentially associated with the attribute dimension, the subject should find one of the two combined task more difficult that the other. Given that he same attribute (pleasant-unpleasant) once is associated with White name and once with Black names, the subject will be faster in the block in which the two concepts are associated.

As far as concerns the possibility to fake the IAT effect, recent studies demonstrate that, if the subject doesn't know how the instrument functions, it is practically impossible for him/her to control consciously his/her responses (Asendorpf, Banse & Mücke., 2002; Banse, Seise & Zerbes, 2001,2001; Egloff & Schmuckle, 2002; Fielder & Bluemke, 2005; Kim, 2003; Steffens, 2004). Naive subjects, first encounters of an IAT, are not able to alter their responses, even if this is explicitly requested and it is explained how to do that. When subjects (Kim, 2003) are instructed to “try to respond slowly” in the compatible condition and to “try to respond quickly” in the incompatible condition, they are more efficient in altering the true results. Another more recent research of Fielder and Bluemke (2005) shows that, if the participants had a previous experience with the IAT, they are able to consciously distort their IAT scores.

The traditional IAT measures associations between concepts (semantic memory), while the method of the present invention measures association between autobiographical memories and intentions. For this reason important modifications have been introduced that render the technique ideal for lie-detection purposes:
stimuli are sentences that may be used to describe virtually any autobiographical event: sentences used have to be short;
the categories are “true”, “false” and “guilty” and “innocen”: sentences referred to the category “true-false” are always true or false for the participant while sentences referred to the categories “guilty/innocent” report two autobiographical memories only one of which is true.

In respect to the traditional IAT, the stimuli (words or pictures) have been modified and substituted with sentences that describe an autobiographical event, for this reason the instrument is termed “autobiographical IAT”.

The Autobiographical Implicit Association Test (aIAT) is the novel variant of the Implicit Association Test (IAT, Greenwald et al., 1998) which has been previously used to assess concepts associations. The method aims to reveal factual knowledge regarding autobiographical events presented in a verbal format. More specifically, with the aIAT it is possible to evaluate which of two autobiographical events is true. In particular this task has shown that criminals have a clearly detectable pattern of associations regarding crimes which can be distinguished by the pattern exhibited by innocent suspects (Sartori, Agosta, Zogmaister, Ferrara, Castiello, 2008).

The aIAT measures the associations a suspect may have between a crime-related verbal description (here defined as “Guilty” sentence) and the logical dimension “True”. First, participants categorize “Guilty” sentences (e.g., “I killed my mother”) and “True” sentences (i.e., sentences that are surely true for the respondent such as “I am in front of a computer”) on the same computer key and “Innocent” sentences (e.g., “I did not kill my mother”) and “False” sentences (i.e., sentences that are surely false for the respondent such as “I am climbing a mountain”) with a different key. Participants, subsequently categorize “Innocent” sentences and “True” sentences on the same computer key and “Guilty” sentences and “False” sentences with the other key. Responses are provided trough a response-box. Please note that only one amongst the “Guilty” and “Innocent” sentences could be true for the respondent. Congruent responses are those in which there is a correspondence in response mode between sentences describing what has really happened to the participants and “True” and sentences describing what has not happened and “False”. Incongruent responses are those in which there is a correspondence between what has really happened and “False” and a correspondence between what has not really happened and “True”. Congruent responses are expected to be faster than incongruent responses. The associations of interest are indexed by means of the difference between reaction time (RT) for the incongruent condition minus reaction time for the congruent condition.

The apparatus 4 of the invention allow to interpret the results and consequently to identify the memory trace and/or the subject's intention. In particular the apparatus 4 comprehend an elaboration unit 8 with visual (monitor) and audio devices 12 (headphones) in order to administer the test to the subject. It includes also at least two response keys 16,20 in order to record the subject's speeded classification (keyboard or mouse or response pad). The unit 8 allows the recording of the reaction times and the memorization of the responses and RTs. Time reaction are recorded with a timer and the responses and the time reaction are recorded in the elaboration unit 8. In particular the software perform a series of analysis of the results acquired by the elaboration unit during the test.

Results are analyzed using two dependent measures; RTs and D-IAT. RT will be submitted to a analysis of variance (ANOVA). The D-IAT index (Greenwald, Nosek & Banaji, 2003) includes a penalty for incorrect trials, and expresses the aIAT effect (the difference in performance between the incongruent and congruent blocks) in terms of the standard deviation of the latency measures. The D-IAT index is calculated by subtracting corrected mean RTs obtained for the incongruent block from those obtained for the congruent block for the two orders of blocks (“4 of diamonds” order and “7 of clubs”) and dividing this difference by the inclusive standard deviation of the two blocks of trials. The algorithm can be summarized in eight steps and is based on RTs collected during blocks 3 and 5 (the structure of aIAT in five blocks has been described previously):

1. Use data from 3^{rd} and 5^{th} blocks

2. Eliminate trials with latencies>10,000 ms and inferior to 150 ms

3. Compute mean of correct latencies for each block;

4. Compute one pooled SD for all trials in 3° and 5° blocks;

5. Replace each error latency with block (computed in Step 3)+600 ms;

6. Average the resulting values for each of the two critical blocks;

7. Compute the differences 5° block-3° block;

8. Divide this difference by its associated pooled-trials SD from Step 4.

Given that the forensic setting regards decisions on an individual and not on a group, it is important to show that the above results hold also in the case of an individual, for this reason we performed a binary logistic regression on the D-IAT. The classification of the subject is made on the base of the D-IAT vale that usually varies form −2 to +2. The classification in one of the two categories is based on the positivity or negativity of the value while the degree of association is based on the absolute value of the index. To evaluate the precision of the method in discriminating between groups a Receiver Operating Characteristic (ROC) analysis (Swets, 1988) is performed. The ROC curve indicates the sensitivity of the D-score in classifying subjects. The area under the ROC curve assumes values between 0 and 1. An area of 0.5 signifies that the two distributions cannot be differentiated and therefore in these circumstances it would not be possible to classify the participants. An area of 1 means that there is no overlap between the two distributions.

The procedure has been validated trough various experiments which are described below. The procedure of validation refers to experimental procedures described in the field literature.

### Card Experiment

Here it is possible to evaluate the efficiency of the aIAT in identifying a selected card. Participants were asked to select one of two playing cards (“4 of diamonds” and “7 of clubs”) and to memorize it in a preliminary consolidation task. After the consolidation task they undergo the aIAT. Stimuli have to classified along the logical dimension “True-False” and as “GUILTY” (sentences referred to the card selected by the participant) or “INNOCENT” (sentences referred to the non-selected card). The congruent block for participants who choose the “4 of diamond” is represented by the block in which true sentences and “4 of diamond sentences” are associated, while for participants who choose the “7 of clubs” it is represented by the block that associates true sentences and “7 of club” sentences. For each block, 3 and 5, 60 trials have been presented.

The aIAT has been proven to be able to classify participants who choose the 4 of diamonds and participants who choose the 7 of clubs with an accuracy of 98%, as proven by the ROC analysis (AUC=0.985). The aIAT outperformed classification accuracy based on the GKT (Ben-Shakhar & Elaad, 2003; AUC=0.80) and fMRI (Langleben et al., 2005; AUC=0.80) for the same test. The aIAT correctly identifies 35 over 37 participants and RTs are lower in the congruent condition (972 ms) than in the incongruent condition (1288 ms), indicating a strong association between the chosen card and the logical category True/False.

### Mock Crime Experiment

Here “Guilty” participants simulated a theft, whereas “Innocent” participants simply read a press report on the same issue. “Guilty” suspects were instructed to enter the office of a teaching assistant and steal a CD-ROM containing a copy of the to-be-done examination for the Neuropsychology course. “Innocent” suspects read a press report on this event. The procedure was similar to the one of the previous experiment except for the different sentences used for the Guilty (e.g. “I stole the CD containing the exam for the course of Neuropsychology”) and Innocent (e.g. “I did not steal the CD”) categories. Also in this case our instrument is able to differentiate between guilty suspects and innocents with an accuracy of 96%, as revealed by the ROC analysis. The aIAT outperformed the GKT (AUC=0.87; Ben-Shakhar & Elaad, 2003) in classification accuracy. RTs of the congruent block (1091 ms) are lower than RTs of the incongruent block (1520 ms).

### Heroin and Cocaine Experiment

Here the aIAT is applied within an ecological setting: the detection of illegal behaviors such as drug usage. We tested subjects with at least 5 years of heroin and cocaine abuse. Half of the participants were administered a version the aIAT that investigated their previous use of cocaine. The other half were administered a version of the test that investigated their previous use of heroin. They had to classify sentences referred to their previous use of drug and classify them as Guilty sentences (“I abused of cocaine”) and Innocent sentences (“I never used cocaine”). Thirteen out of 14 participants where correctly identified as drug users.

### Autobiographical Memory Experiment

It might be argued that the sentences used in heroin and cocaine experiment were not tapping into autobiographical memories, but rather describing participants' characteristics. To ascertain the efficiency of the aIAT in detecting single autobiographical events limited in time and space, participants were asked to report a personal experience. Participants had to describe their last vacation and then they were administered the aIAT with sentences referred to their real vacation (e.g. “Vacation in Paris”) and sentences referred to a false vacation (e.g. “Vacation in London”). Results show that for 18 out of 20 of the participants we correctly identified the real event based on the double-categorization block in which they were fastest. Time reactions of the block in which the true vacation is associated with true sentences are lower than RTs in the block in which sentences referred to the false vacation are associated with true sentences.

### Suspension of Driving License for Drunk Driving Experiment

A possible problem related to the previous experiments is that participants were not exposed to the high level of stress typical of an investigative setting and they would not experience direct advantages from faking. Indeed, an important challenge for experimental studies of deception is to use a valid setting comparable to real situations where participants may lie or conceal spontaneously. Therefore we decided to run an experiment in which participants were highly motivated at passing the test. The main feature of the experimental group was that all participants had their driving license suspended for driving with excessive alcohol blood level. The aIAT was included as part of the compulsory medical and psychological assessment requested for the reinstatement of the driving license. Participants were made to believe that driving license reinstatement depended on the aIAT outcome. “TRUE” and “FALSE” sentences were the same as for all the previous experiments. “GUILTY” sentences were 5 sentences describing the illegal act. “INNOCENT” sentences were sentences describing that the driver was never caught drunk by the police. The experimental group (“Guilty” participants) was expected to show an association between “TRUE” sentences and “GUILTY” sentences (and between “FALSE” and “INNOCENT” sentences) whereas the control group (“Innocent” participants) was expected to show the reverse pattern. The ROC analysis yielded an AUC=0.91 in distinguishing between guilty and innocents participants.

### Confirmed Crimes

The aIAT has been administered to individuals who were found guilty after having confessed their crime and classified as mentally insane on the basis of a forensic psychiatric assessment. Both were under medication and were examined in a Forensic Mental Hospital. The first examinee (D.E.), attempted to kill his two sons. The second examinee (C.S.) was found guilty of killing his mother. For each criminal a personalized aIAT was built with “GUILTY” sentences describing the crime and “INNOCENT” sentences concerned with the denial of the crime. Administration of the aIAT to the first criminal (D.E.) revealed a strong association between “True” and “Guilty” sentences., as well as the administration to the second examinee.

The administration of the aIAT showed faster responses, in terms of RTs, in the congruent block (in which true sentences and Guilty sentences are associated) in respect the incongruent block (in which false sentences and Innocent sentences are associated).

Results of these experiments clearly show that the aIAT is an accurate instrument to identify the guilty knowledge and that it has an higher accuracy in respect to the other methods. It is also flexible and can be used in order to verify the existence of any memory. Another advantage over the other methods is the procedure to control for the counter-measures, all the other methods are susceptible to countermeasures.

Faking a test means to produce different results from the real ones. Effective countermeasures are known for almost every lie-detection technique. The first investigation about detection of countermeasures in polygraphic lie-detection goes back to Benussi (1914) who also introduced the first respiratory-based lie-detection technique. Countermeasures to the CQT have long been known. Most attempt to increase the response of a subject to the control questions using physical (e.g., biting the tongue or pressing the toes to the floor) or mental (e.g., counting backward by 7) techniques (Honts, Raskin, & Kircher, 1994). A number of experiments (Ben-Shakhar & Dolev, 1996; Elaad & Ben-Shakhar, 1991; Honts, Devitt, Winbush, & Kircher, 1996; Honts, Hodes, Raskin, 1985; Honts, Raskin, & Kircher, 1994; Kubis, 1962) have indicated that it is possible, indeed quite easy, to train effective countermeasures to available lie-detection techniques. All these procedures consist in preparing guilty examinees for a polygraph examination (either CQT, or GKT) in such a way that with a high probability they will be found truthful. This can be done by adopting some rather simple techniques (that can be picked up with little effort), which can cause very strong reactions to the control questions.

These techniques rely either on the use of physical means (such as biting one's tongue), or mental means (calling to mind an exciting or frightening event, or engaging in mental activities that require effort, such as subtracting 7 from 100 multiple times) each time a control question is asked. A series of experiments conducted (Honts et al., 1994) demonstrated that the use of such countermeasures could be most effective. They showed in different experiments that the error rate produced by polygraphers testing “guilty” examinees who were using countermeasures ranged between 50 and 70 percent. Clearly, countermeasures may increase false-negative outcomes (guilty suspects classified as “innocents”). It should be pointed out that the type of countermeasures that are most detrimental for all psychophysiological-detection techniques are mental countermeasures, because mental manipulations cannot be detected even by the most experienced examiners. The same mental countermeasures (counting backwards) can be used while undergoing the fMRI technique, in fact a difficult calculation activates the same neural network of deception and if a calculation is performed while asked a control question, this response is the same of the critical question and no differences are noticed.

The invention comprehend a procedure for the evaluation of the faking described below. In order to guarantee a practical use of the invention is necessary to show that the method allow the identification of the actual result even if a subject, instructed, is trying to fake the results. Two studies have been conducted to validate the procedure:

1. malingered depression

2. faking of autobiographical memory.

Three group of subject have been run: non faking participants, naive-faking participants and instructed faking participants. Participants for the non-faking group received the standard aIAT instructions (i.e., they were requested to categorize the sentences as indicated by the labels by pressing the appropriate keys as fast and accurately as possible), participants for the naive-faking group were further asked to do their best as to hide their true autobiographical memory to the experimenter (Fiedler & Bluemke, 2005) but they were not instructed on how to fake the test. Participants for the instructed-faking group were instructed to slow down in the congruent block and speed up in the incongruent block (Kim, 2003).

Malingering is frequent in a forensic context in order to obtain economical or personal advantages, for this reason it is important to evaluate the truthfulness of the referred symptoms using the aIAT. The Diagnostic Manual defines malingering as follow: “intentional production of false or grossly exaggerated physical or psychological symptoms, motivated by external incentives such as avoiding military duty, avoiding work, obtaining financial compensation, evading criminal prosecution, or obtaining drugs”. The two critical points are: the intentionality (voluntary production) and the aim (to obtain an advantage).

The three groups of participants were:
control group: 27 participants scored at the CBA scale as non depressed (score inferior to 4)
naive faker group: 27 participants scored as non depressed at the CBA scale, they received the instruction that they had to malinger depression symptoms while performing the aIAT (all of them were student and were told that they would have received an higher degree on the final course if they were able to malinger depression)
instructed faker group: 27 participant instructed to use countermeasures in order to malinger depression at the aIAT.

The aIAT is used here as a diagnostic tool for malingered depression. It consists in measuring reaction times in order to evaluate whether sentences describing depressive symptoms are true or false for the respondent. In one block, participants categorize sentences describing DEPRESSIVE condition and TRUE sentences on the same computer key and sentences describing a ‘HEALTHY’ condition and FALSE sentences with the other computer key. In a later block, the tasks are reversed and participants categorize sentences describing the “HEALTHY” condition and TRUE sentences on the same computer key and sentences describing the DEPRESSIVE condition and FALSE sentences on another computer key. An overall IAT score is computed by taking the difference between the average response times to the two test stages. The faster block will indicate either the depressive condition or the not depressive condition is the most strictly associated with TRUE sentences. The results show that the control subject are faster in the block associating true sentences and sentences belonging to the Healthy condition category. Regarding the naive faker group:

1. 7 out of 27 fake the results

2. 20 out of 27 fail to fake the results

but with a series of indexes described later it is possible to identify people that succeed in faking the aIAT. The instructed fakers succeed in faking the results, they are able to invert their congruency, but with a series of indexes described later it is possible to identify people that succeed in faking the aIAT with an accuracy of 92%. The index have been classified in different families:
1. global indexes (mean global RTs);
2. slow-down indexes consisting in the subtraction between the average RT of the fastest block (between 3 or 5) and single tasks that are directly connected to the fastest one in terms of motor response (1 and 2 or 1 and 4, respectively);
3. Ratio 1: consisting in the ration between the mean block 3 and 5 and the mean blocks 1, 2 and 4;
4. Ratio 2: consisting in the ratio between the average RT of the fastest block (between 3 or 5) and single tasks that are directly connected to the fastest one in terms of motor response (1 and 2 or 1 and 4, respectively).

All the indexes have been calculated for both latencies between 150 and 10000 ms and between 300 and 3000 ms (Greenwald et al., 2003), RTs inferior to 300 ms have been calculated as 300 ms and RTs superior to 3000 ms have been calculated as 3000 ms.

Another experiment has been run in order to verify the possibility to fake the aIAT conducted to detect autobiographical memories.

Three groups of participants have been used:

control group: 15 participants
naive faker group: 10 participants that received the instruction to fake the aIAT.
instructed faker group: 15 participants, they were told to speed up in the incongruent block and slow down in the congruent block.

Sentences belonging to two logical categories true and false and sentences describing two autobiographical events with only one of them being true (e.g., Christmas in Paris vs. Christmas in London) were used (true and false autobiographical events were specific for each participants and collected preliminarily through a questionnaire). The aIAT is accomplished by requiring the respondent to complete five blocks of speeded categorization trials. Participants are requested to classify the sentences by pressing one of two labeled keys, one positioned on the left of the keyboard (e.g., “A”) and one situated on the right of the keyboard (e.g., “L”). Sentences are presented on the center of the monitor and two reminder labels are positioned, one on the left and one on the right of the monitor. These two labels show the name of the categories that must be used in order to classify each sentence. Within these five blocks two are the critical blocks which require the double categorization of an autobiographical event (e.g., Christmas in Paris or Christmas in London) with certainly true events. In Block 1 (20 trials) participants had to classify certainly true or false sentences, by pressing the left key to classify certainly true sentences (5 different sentences; e.g. I am in front of a computer) and the right key to classify certainly false sentences (5 different sentences; e.g. I am in front of a television). In Block 2 (20 trials) participants had to classify the autobiographical sentences. They pressed the left key to classify real autobiographical-event sentences (5 sentences; e.g., I saw the Tour Eiffel) and the right key to classify the false autobiographical-event sentences (5 sentences; e.g., I saw the Big Ben). In Block 3 (60 trials), the left key was used to classify both certainly true sentences and real autobiographical event sentences, whereas the right key was used to classify both false sentences and false autobiographical-event sentences (congruent block). In Block 4 (40 trials) the left key was used to classify false autobiographical-events sentences, whereas the right key was used to classify real autobiographical-event sentences. Finally, in Block 5 (60 trials), participants had to classify with the left key both true and false autobiographical-event sentences, and with the right key they had to classify false and real autobiographical-event sentences (incongruent block). Because the pairing of a truly autobiographical event with certainly true sentences should facilitate the response, the specific pattern of response times (RTs) in the two blocks (3 and 5) indicates which autobiographical event is true and which autobiographical event is false. Results show that the indexes described before allow an optimal classification of the instructed fakers (100% correct classification).

As is possible to notice, the present invention is able to overcome the previous highlighted problems related with use of the lie-detector, in particular:
1. invention has the highest accuracy reported;
2. can be web administered and it has a short administration (10 minutes);
3. algorithmic scoring, no interpretation requested;
4. accuracy superior to 92% and superior to the other methods available;
5. the method can identify not only autobiographical memories but also intentions;
6. the method has different field of application, other than lie-detection: security, intelligence, investigation, marketing and work selection.

In particular the aIAT can be a useful instrument in the field of security and investigation when used to identify the actual autobiographical memory.

In the security field, the primary aim regards the national security and the main objective is to control the entrance at the frontiers. The aIAT is an invention that can be used to examine suspects at arrivals at the airport. The web version of the invention will allow to examine the suspect in his/her State at the Consulate, in order to verify criminal past behavior and “at risk” future intentions. Its rapidity allow the application in fields where the polygraph application would not be possible.

During investigations the method that we propose is an optimal method because it permit to verify the investigative hypothesis, as the previous polygraph.

It is also possible to use the invention in the forensic field. At present, the forensic rules in the west Countries limit the direct examination of the suspect, in fact he/she can refuses to being analyzed (e.g. administration of psychological tests, or specimen analysis). The innocent suspect has advantages by being administered the aIAT, given that this objective instrument can show his/her innocence and validate his/her version of the facts.

In the scientific and research fields “lie detection techniques” are know also for their deterrent power. It is thought that the administration of the lie detection techniques can prevent illegal activities, this sentence is a valid supposition but need to be further investigated.

Another important application field regards insurances, we report the example of the numerous thefts and in particular in the jewelry field, where insurances companies request the conveyor (who was robbed) to pass the lie detector. Our methods find here another ideal application.

Two other important applications regard work selection, where it is important to identify the perfect candidate for a determinate position and to evaluate his/her moral integrity (e.g. in the case of a branch company). And the marketing field, where it is important to foresee the intended purchases. At present the “buyer's intention” is evaluated on a Likert scale with 7 points, this technique is not reliable in fact it is possible to fake the test (for various reasons), while the aIAT evaluates the buyer's intention implicitly not allowing the intentional faking and producing an accurate measure of the buyer's intention.

Another interesting field is the “intention detection”. In a series of experiments, 30 participants were preliminary requested to fill in a questionnaire in which they had to rate whether an intention was true or false. Then, we asked participants to classify sentences describing possible future events and true and false sentences by pressing one of two response keys. Responses were faster (p<0.001) when sentences related to true intentions shared the same response key with other sentences reporting true events (congruent block) and slower when sentences related to true intentions shared the same response key with sentences reporting false events (incongruent block).

The apparatus and the method allow the identification of the malingerer and fakers.

A man skilled in the art can modify and produce new variants of the method and the apparatus of the present invention in order to satisfy specific demands; all of them are comprehended in the invention as defined in the following claims.

I claim:

1. Apparatus for the measurement of a subject's memory trace and for the intention prediction, comprising
a unit of elaboration comprising a software that implements a series of test,
visual means and/or audio-visual means to administer the test to the subject, characterized in that the apparatus comprises
at least a first and a second key for the input, in the unit of elaboration, of the responses to the test
means for measuring the reaction time of the responses
means for storing the subjects responses and the relative reaction time and in that
the software of the apparatus comprises a plurality of tests divided in different phases, the tests being based on sentences describing concepts or actions to be classified, pressing respectively the first and second key, wherein
a first phase comprises the classification, by the subject, of innocent and guilty sentences, in the guilty sentences the subject declaring to have committed an action, while in the innocent sentences the subject declaring not to have committed an action, the classification comprising the pressing of the first and second key answering the sentences subdued to the subject, the apparatus storing the response and the relative reaction time of the first phase,
a second phase comprises the classification of true and false sentences, the true sentences relating to facts absolutely true and the false sentences relating to facts absolutely false, the apparatus storing the response and the relative reaction time of the second phase,
a third phase comprises the classification of true and guilty sentences with the same key and of false and innocent sentences with another key, the apparatus storing the response and the relative reaction time of the third phase,
a fourth phase comprises the classification of guilty and innocent sentences, using the keys of the first phase in a reversed manner, the apparatus storing the response and the relative reaction time of the fourth phase,
a fifth phase comprising the combinations of the sentences of the third phase, the classification of guilty and false sentences with the first key and of innocent and true sentences with the second key, the apparatus storing the responses and the relative reaction time of the fifth phase.

2. Apparatus according to claim 1, wherein the unit of elaboration comprises a software which eliminates the reaction time with latencies inferior to 150 ms and superior to 10000 ms, calculates the standard deviation, using latencies of the third and fifth phases, calculates the mean latencies of the correct classifications in the third and in the fifth phases, substitutes the reaction times of the wrong trials with the mean of the corresponded phase added of a time of 600 ms, calculates the mean of the reaction times of the critical phases (corrected for the errors), calculates the difference of the reaction times of the fifth and third phases, divides the result for the standard deviation, gets an index called IAT-d through which is possible to classify the subject as belonging to one of two categories in relation to the memory trace identified by the apparatus.

3. Apparatus according to claim 2, in which the elaboration unit comprises a software that calculates indexes to highlight fakers subjects, said indexes comprising
the global mean reaction time, greater for subject that try to fake their data,
slow down indexes, equal to the difference between the reaction time of the phase defined as congruent and the mean of previous phases, that directly anticipate the congruent phase,
ratio index 1, equal to the ratio between the mean of the double classification phases (third and fifth phases) and the mean of reaction times of single phases (first, second and fourth phases) classification phases,
ratio index 2, equal to the ratio between the congruent phase and the previous single classification phase.

4. Apparatus according to claim 1, in which the elaboration unit comprises a software that calculates indexes to highlight fakers subjects, said indexes comprising
the global mean reaction time, greater for subject that try to fake their data,
slow down indexes, equal to the difference between the reaction time of the phase defined as congruent and the mean of previous phases, that directly anticipate the congruent phase,
ratio index 1, equal to the ratio between the mean of the double classification phases (third and fifth phases) and the mean of reaction times of single phases (first, second and fourth phases) classification phases,
ratio index 2, equal to the ratio between the congruent phase and the previous single classification phase.

5. Method for the measurement of a memory sign of a subject and for predicting intention, comprising the steps of:
administer to the subject, by means of visual and audio/visual means a series of tests, in different phases, the tests being based on sentences describing concepts or actions to be classified by pressing a first and a second key in response to the test, wherein
the first phase comprises the classification of innocent and guilty sentences, the storing of the answers of the subject, by pressing one of the two keys, the storing of the reaction times of the subject, in the guilty sentences the subject declaring to have committed an action, while in the innocent sentences the subject declaring not to have committed an action,
the second phase comprises the classification of true and false sentences, the storing of the answers and of the reaction times of the subject, the true sentences relating to absolutely true facts and the false sentences relating to absolutely false facts,
the third phase comprising the classification of true and guilty sentences with the same first key and of false and innocent sentences with the other key, the apparatus storing the answers and reaction times of the subject,
the fourth phase comprising the classification of guilty and innocent sentences, keys being reversed with respect to the first phase, the storing of the answers and reaction times of the subjects,
the fifth phase comprising the classification of guilty and false sentences with the first key and of innocent and true sentences with the second key, the storing the answers and reaction times of the subject.

6. Method according to claim 5, comprising the phases of
eliminating latencies inferior to 150 ms and superior to 10000 ms,
calculating the standard deviation using latencies of the third and fifth phases;
calculating the mean latencies of the correct trials in the third and in the fifth phases;
substituting the reaction times of the wrong trials with the mean of the corresponded phase added of a penalty of 600 ms;
calculating the mean of the corrected third and fifth phases;
calculating the difference between the fifth and the third phase, dividing the result for the standard deviation and getting an index called IAT-d through which it is possible to classify the subject in one of two categories, in relation to the memory trace of the subject.

7. Method according to claim 6, comprising the phases of calculating indexes to highlight fakers subjects, said indexes comprising
global mean reaction time, greater for subjects that try to fake their data,
slow down index, equal to the difference between the reaction times of the congruent phase and the mean of the phases preceding the congruent phase,
ratio index 1, equal to the ratio between the mean of the double classification phases (third and fifth phases) and the mean of reaction times of single phases (first, second and fourth phases) classification phases,
ratio index 2, equal to the ratio between the congruent phase and the previous single classification phase.

8. Method according to claim 5, comprising the phases of calculating indexes to highlight fakers subjects, said indexes comprising
global mean reaction time, greater for subjects that try to fake their data,
slow down index, equal to the difference between the reaction times of the congruent phase and the mean of the phases preceding the congruent phase,
ratio index 1, equal to the ratio between the mean of the double classification phases (third and fifth phases) and the mean of reaction times of single phases (first, second and fourth phases) classification phases,
ratio index 2, equal to the ratio between the congruent phase and the previous single classification phase.
